(19) United States
(12) Patent Application Publication
(10) Pub. No.: US 2005/0273001 A1
(43) Pub. Date: Dec. 8, 2005

(54) MRI display interface for medical diagnostics and planning

(75) Inventors: Kathleen Schmainda, Elm Grove, WI (US); Robert W. Prost, Nashotah, WI (US); John L. Ulmer, Brookfield, WI (US); Edgar DeYoe, Delafield, WI (US)

Correspondence Address: QUARLES & BRADY LLP, 411 E. WISCONSIN AVENUE, SUITE 2040, MILWAUKEE, WI, 53202-4497, US

(73) Assignee: The MCW Research Foundation

(21) Appl. No.: 11/143,859

(22) Filed: Jun. 2, 2005

Related U.S. Application Data
(60) Provisional application No. 60/577,320, filed on Jun. 4, 2004.

(51) Int. Cl. A61B 5/05

(52) U.S. Cl. 600/411

(57) ABSTRACT

An interactive display interface may import fMRI images, DTI images, MRS images and perfusion images of the brain and selectively display them on top of one another and aligned with an anatomical image of the brain. The transparency of each layered image can be adjusted or turned on or off to assist in the planning of a treatment strategy for a brain tumor or other diseased region in the brain.

### CROSS-REFERENCE TO RELATED APPLICATIONS

This application is based on U.S. Provisional Patent Application Ser. No. 60/577,320 filed on Jun. 4, 2004 and entitled “MRI DISPLAY INTERFACE FOR MEDICAL DIAGNOSTICS AND PLANNING”.

### STATEMENT REGARDING FEDERALLY SPONSORED RESEARCH

This invention was made with government support under Grant No. NIH CA82500 and Grant No. NIH 5 R01 EY13801 awarded by the National Institute of Health. The United States Government has certain rights in this invention.

### BACKGROUND OF THE INVENTION

The field of the invention is magnetic resonance imaging (MRI), and particularly, the imaging of tumors in the human brain.

In the United states, approximately 17,000 new patients are diagnosed each year with a primary intracranial neoplasm. Approximately 60% of these tumors are malignant, and gliomas are the most common type. Although there is a wide variability in life expectancy for patients with the various subtypes of gliomas, their prognosis is generally poor. This is especially true for those with high-grade gliomas, in spite of treatment modalities such as surgery, radiation therapy and chemotherapy.

Magnetic resonance imaging (MRI) methods have become the imaging standard for the depiction and detection of brain tumors. Such MRI methods include perfusion imaging as described in co-pending U.S. patent application Ser. No. 09/861,220, which produce images of relative cerebral blood volume (rCBV) that differentiate histologic tumor types and provide information to predict glial tumor grade. Perfusion imaging also produces images of cerebral blood flow, vessel and tissue blood transit times and vascular morphology. Other MRI imaging techniques include diffusion tensor imaging, (DTI) as described in U.S. Pat. No. 6,526,305 which produce images that enables one to observe the molecular organization of tissues. Magnetic resonance spectroscopy (MRS) produces images of metabolites indicative of cell health as described in U.S. Pat. No. 6,320,381.

MRI has also become a preferred technique for imaging brain functions. Functional MRI (fMRI) acquires a series of brain images while the subject is performing a prescribed task or is subjected to a prescribed stimulus. Such a method is described in U.S. Pat. No. 5,603,322 and the images which are produced depict the anatomical structure of the brain with those regions that function in response to the activity or stimulus shown in color. Such images are an important component of disease assessment since they can measure the functional impact of a detected tumor and they can estimate the functional improvement that will result from various treatment strategies. See for example U.S. Pat. No. 6,430,431 which employs fMRI to indicate locations in a subject's field of view which will be impaired by disease or intervention at locations in the subject's brain.

Currently there is no method of depicting these images in such a way that the spatial relationships between the many imaged physiological and functional parameters can be assessed for surgical or radiation treatment planning. The images are separately acquired by a neuroradiologist who typically describes what is seen in each image using verbal communications and perhaps a copy of each image. Treatment planning is thus done using a series of written reports and perhaps a corresponding series of images.

### SUMMARY OF THE INVENTION

The present invention is a display interface that enables a series of images acquired using a variety of different methods to be selectively displayed in such a manner that the physiological and functional parameters which they depict are spatially registered among themselves and the normal surrounding brain and focal brain pathologies.

To address the problems described above, we propose a system for image-guided-diagnosis (IGD) and treatment (IGT) of tumors and other focal pathologies involving human cerebral cortex and related structures. FMRI in combination with a suite of uniquely efficient test stimuli are used to map functionally responsive brain tissue near a tumor site. The fMRI data are then combined with conventional MR images and other physiological imaging data (eg. DTI, rCBV, perfusion, spectroscopy) to allow the physician to visualize the brain pathology, anatomy and function and then plan invasive treatment strategies that maximally reduce the tumor yet avoid or minimize damage to eloquent tissue critical for behavioral function. A unique display technology allows the physician to estimate the effects of a proposed treatment regime on the patient's abilities. Then using a second unique display technology, the patient can experience a simulation of the potential impact of the proposed treatment on his/her function, at least for sensory systems such as vision and touch. With refinements, similar risk-benefit analyses for language and motor function will be possible. In short, the system provides a suite of interactive tools to assist the physician and patient in selecting the most appropriate treatment options to optimize therapeutic effect while maximally preserving function and quality of life.

A general object of the invention is to enhance the capability of clinicians to plan therapy for brain tumors and other diseases. Because treatments such as surgery and radiation therapy of brain tumors have been shown to increase survival, preserve quality of life, and preserve normal brain functioning, the invention is designed to enhance these positive prognostic therapeutic maneuvers. The interface will not only allow for more accurate pre-treatment planning, but will allow for virtual treatments to be carried out prior to actual treatments, with the capability of demonstrating likely functional neurologic deficits that will occur with the particular treatment approach.

The invention is specifically designed to visually display critical spatial relationships among various physiological MRI and non-MRI data sets, in relation to pathological processes and normal tissues in patients with diseases of brain or other organs. The interface is designed to aid clinicians in the diagnosis, treatment, and management of such patients. The invention also has considerable utility in research of various pathological conditions as well as the development and evaluation of new drugs and treatment strategies. The essence of the discovery is the ability to visually display vital spatial and functional relationships among multiple physiologic imaging data sets in relation to normal tissue and pathology, and the ability to manipulate these data sets to best optimize diagnosis, treatment planning, rehabilitation and patient management.

The display is composed of a set of image windows and associated menus of various anatomic and physiologic imaging data sets, viewpoint alternatives, and user preferences similar to existing PACS capabilities. However, unlike current PACS systems, the device can be utilized to manipulate and compare multiple two-dimensional, three-dimensional and four-dimensional (spatial and time dimensions) image data sets simultaneously and in proper spatial alignment. A menu of various physiologic imaging data sets allows the user to select any, or all, physiologic parameters to be superimposed upon existing anatomic and morphologic imaging sets. The device also provides an interface between these anatomically oriented views and additional non-anatomical displays that allow rapid, intuitive, interpretation of brain function as revealed by the physiologic imaging data (functional field maps). By linking the different anatomical and functional data sets in this way, the system provides unique capabilities for assessing the relationships between a site of pathology and surrounding tissue function and allows for treatment planning through virtual surgery, virtual radiation or other localized therapies (such as but not limited to radioactive seed implantation, cryo-therapy, therapeutic radio-frequency ablation, tissue implants).

### BRIEF DESCRIPTION OF-THE DRAWINGS

FIG. 1A is a block diagram of an MRI system used to acquire images employed by the preferred embodiment of the invention;

FIG. 1 is a pictorial view of a preferred embodiment of an interactive physiologic imaging interface which employs the present invention. Toggle functions display on or off individual physiologic maps onto existing anatomic sequences in multiple planes. Any data set can be turned on or off and thresholds for that data can be individualized for a given parameter. Sub-parameter toggle switch between data sets for a given technique, such as between motor and language fMRI. Translucency bars determine the =opaqueness of any parameter, superimposed onto anatomic and pathologic imaging information obtain with standard imaging. Adjacent lower toggle (outlined box) outlines (i.e completely translucent) data sets of interest;

FIG. 2 is a pictorial view of the interface showing that fMRI and DTI data sets have been selected, showing sensori-motor cortex and white matter locations in relation to a tumor. Functional system and white matter orientation designated in the upper corners of the image respectively, and in matching colors respectively;

FIG. 3 is a pictorial view of the interface in which green (anterior-posterior) white matter fibers have been unselected (toggled off) (white arrow), as they are not close to the tumor and unnecessarily cover information on the standard imaging sequence;

FIG. 4 is a pictorial view of the interface in which all physiological maps have been selected, but the maps cover much important morphological data on the standard sequence;

FIG. 5 is a pictorial view of the interface in which toggles have been selected to make MRS and rCBV data sets completely translucent and color outlined (arrows), to better illustrate spatial relationships among physiological data and the tumor;

FIG. 6 is a pictorial view of the interface in which all data sets have been outlined and colors correspond to those in upper corners of the images;

FIG. 7 is a pictorial view of the interface in which unnecessary data (fMRI, DTI) have been removed (arrows), leaving only those outlines deemed important (rCBV, MRS) for a given stage of treatment;

FIGS. 8A and 8B are pictorial views of the interface in which upper fMRI toggle (arrow) switches between sub-components of fMRI data (i.e. motor and language functional maps), as designated in the upper left corner of the image;

FIGS. 9A and 9B are pictorial views of the interface in which MRS data has been selected (arrow)(A). Growth vectors have been derived from that data and displayed by selecting the upper MRS toggle (arrow) (B);

FIG. 10 is a pictorial view of the interface demonstrating how virtual surgery can be performed, based on fMRI data. Outlining pathology (green outline—left) demonstrates corresponding portion of the visual field map that may be at risk (dotted outline—right);

FIGS. 11A and 11B are pictorial views also demonstrating virtual surgery. The planned resection (shaded triangle on brain) automatically indicates the associated portion of the visual field at risk (A), and alerts the user of the risk of injury to critical foveal (central, dotted white outline) vision functions (B); and

FIG. 12 is a block diagram of the software modules of a system that includes an interactive display interface that performs the interface functions shown in FIGS. 1-11.

### DETAILED DESCRIPTION OF THE PREFERRED EMBODIMENT

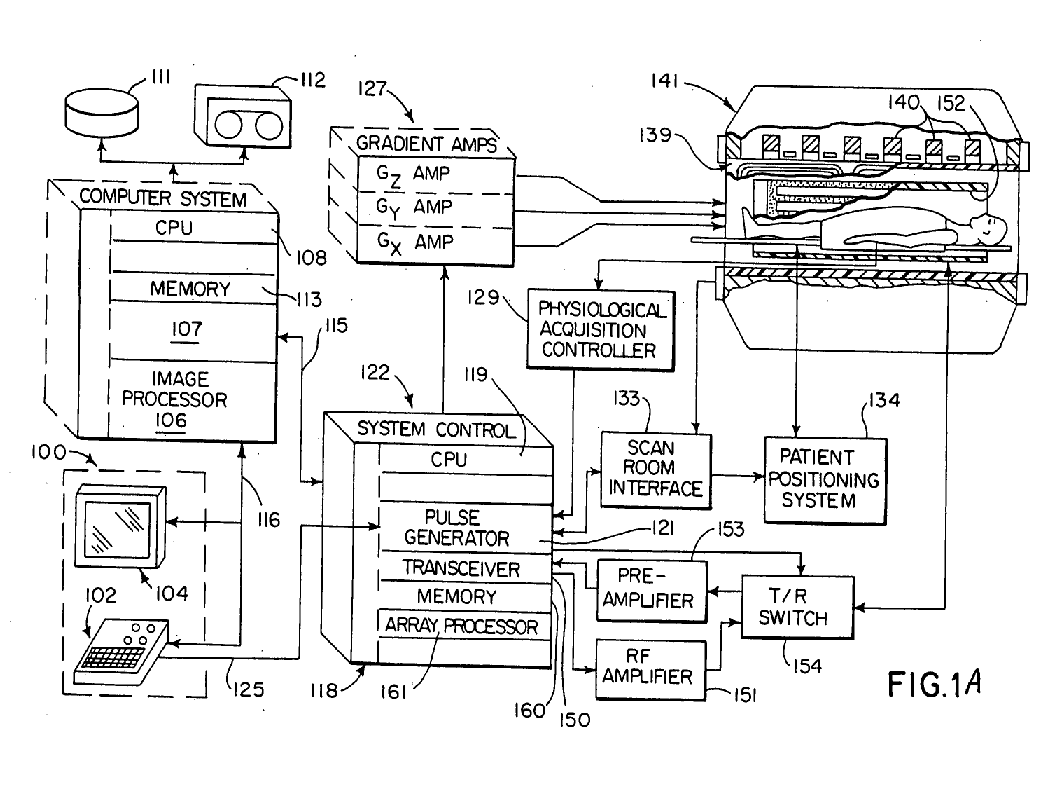

Referring first to FIG. 1A, there is shown the major components of a preferred NMR system which incorporates the present invention. The operation of the system is controlled from an operator console 100 which includes a console processor 101 that scans a keyboard 102 and receives inputs from a human operator through a control panel 103 and a plasma display/touch screen 104. The console processor 101 communicates through a communications link 116 with an applications interface module 117 in a separate computer system 107. Through the keyboard 102 and controls 103, an operator controls the production and display of images by an image processor 106 in the computer system 107, which connects directly to a video display 118 oh the console 100 through a video cable 105.

The computer system 107 is formed about a backplane bus which conforms with the VME standards, and it includes a number of modules which communicate with each other through this backplane. In addition to the application interface 117 and the image processor 106, these include a CPU module 108 that controls the VME backplane, and an SCSI interface module 109 that connects the computer system 107 through a bus 110 to a set of peripheral devices, including disk storage 111 and tape drive 112. The computer system 107 also includes a memory module 113, known in the art as a frame buffer for storing image data arrays, and a serial interface module 114 that links the computer system 107 through a high speed serial link 115 to a system interface module 120 located in a separate system control cabinet 122.

The system control 122 includes a series of modules which are connected together by a common backplane 118. The backplane 118 is comprised of a number of bus structures, including a bus structure which is controlled by a CPU module 119. The serial interface module 120 connects this backplane 118 to the high speed serial link 115, and pulse generator module 121 connects the backplane 118 to the operator console 100 through a serial link 125. It is through this link 125 that the system control 122 receives commands from the operator which indicate the scan sequence that is to be performed.

The pulse generator module 121 operates the system components to carry out the desired scan sequence. It produces data which indicates the timing, strength and shape of the RF pulses which are to be produced, and the timing of and length of the data acquisition window. The pulse generator module 121 also connects through serial link 126 to a set of gradient amplifiers 127, and it conveys data thereto which indicates the timing and shape of the gradient pulses that are to be produced during the scan. The pulse generator module 121 also receives patient data through a serial link 128 from a physiological acquisition controller 129. The physiological acquisition control 129 can receive a signal from a number of different sensors connected to the patient. For example, it may receive ECG signals from electrodes or respiratory signals from a bellows and produce pulses for the pulse generator module 121 that synchronizes the scan with the patient's cardiac cycle or respiratory cycle. And finally, the pulse generator module 121 connects through a serial link 132 to scan room interface circuit 133 which receives signals at inputs 135 from various sensors associated with the position and condition of the patient and the magnet system. It is also through the scan room interface circuit 133 that a patient positioning system 134 receives commands which move the patient cradle and transport the patient to the desired position for the scan.

The gradient waveforms produced by the pulse generator module 121 are applied to a gradient amplifier system 127 comprised of Gₓ, G_{y} and G_{z} amplifiers 136, 137 and 138, respectively. Each amplifier 136, 137 and 138 is utilized to excite a corresponding gradient coil in an assembly generally designated 139. The gradient coil assembly 139 forms part of a magnet assembly 155 which includes a polarizing magnet 140 that produces a polarizing field that extends horizontally through a bore. The gradient coils 139 encircle the bore, and when energized, they generate magnetic fields In the same direction as the main polarizing magnetic field, but with gradients Gₓ, G_{y} and G_{z} directed in the orthogonal x-, y- and z-axis directions of a Cartesian coordinate system. That is, if the magnetic field generated by the main magnet 140 is directed in the z direction and is termed BO, and the total magnetic field in the z direction is referred to as B_{z}, then Gₓ∂B_{z}/∂x, G_{y}=∂B_{z}∂y and G_{z}=∂B_{z}/∂z, and the magnetic field at any point (x,y,z) in the bore of the magnet assembly 141 is given by B(x,y,z)=B_{O}+GₓX+G_{y}yG_{z}z. The gradient magnetic fields are utilized to encode spatial information into the NMR signals emanating from the patient being scanned. Because the gradient fields are switched at a very high speed when an EPI sequence is used to practice the preferred embodiment of the invention, local gradient coils are employed in place of the whole-body gradient coils 139. These local gradient coils are designed for the head and are in close proximity thereto. This enables the inductance of the local gradient coils to be reduced and the gradient switching rates increased as required for the EPI pulse sequence. For a description of these local gradient coils which is incorporated herein by reference, see U.S. Pat. No. 5,372,137 issued on Dec. 13, 1994 and entitled “NMR Local Coil For Brain Imaging”.

Located within the bore 142 is a circular cylindrical whole-body RF coil 152. This coil 152 produces a circularly polarized RF field in response to RF pulses provided by a transceiver module 150 in the system control cabinet 122. These pulses are amplified by an RF amplifier 151 and coupled to the RF coil 152 by a transmit/receive switch 154 which forms an integral part of the RF coil assembly. Waveforms and control signals are provided by the pulse generator module 121 and utilized by the transceiver module 150 for RF carrier modulation and mode control. The resulting NMR signals radiated by the excited nuclei in the patient may be sensed by the same RF coil 152 and coupled through the transmit/receive switch 154 to a preamplifier 153. The amplified NMR signals are demodulated, filtered, and digitized in the receiver section of the transceiver 150.

The transmit/receive switch 154 is controlled by a signal from the pulse generator module 121 to electrically connect the RF amplifier 151 to the coil 152 during the transmit mode and to connect the preamplifier 153 during the receive mode. The transmit/receive switch 154 also enables a separate local RF head coil to be used in the transmit and receive mode to improve the signal-to-noise ratio of the received NMR signals. With currently available NMR systems such a local RF coil is preferred in order to detect small variations in NMR signal. Reference is made to the above cited U.S. Pat. No. 5,372,137 for a description of the preferred local RF coil.

In addition to supporting the polarizing magnet 140 and the gradient coils 139 and RF coil 152, the main magnet assembly 141 also supports a set of shim coils 156 associated with the main magnet 140 and used to correct inhomogeneities in the polarizing magnet field. The main power supply 157 is utilized to bring the polarizing field produced by the superconductive main magnet 140 to the proper operating strength and is then removed.

The NMR signals picked up by the RF coil are digitized by the transceiver module 150 and transferred to a memory module 160 which is also part of the system control 122. When an entire array of data has been acquired in the memory modules 160, an array processor 161 operates to Fourier transform the data into an array of image data. This image data is conveyed through the serial link 115 to the computer system 107 where it is stored in the disk memory 111. In response to commands received from the operator console 100, this image data may be archived on the tape drive 112, or it may be further processed by the image processor 106 and conveyed to the operator console 100 and presented on the video display 118 as will be described in more detail hereinafter.

While the present invention may be embodied in the above-described MRI system, it is contemplated that it will also be embodied in a separate work station. The workstation would typically be located elsewhere and be connected to an institution intranet from which it downloads DICOM images directly from the MRI system, or more probably a PACS system which stores medical images from a number of different medical imaging systems. The workstation may also be a free-standing system that simply receives image data from an MRI system. For most situations, the system only needs a synchronization pulse from the MRI system to trigger the behavioral testing sequences used for fMRI.

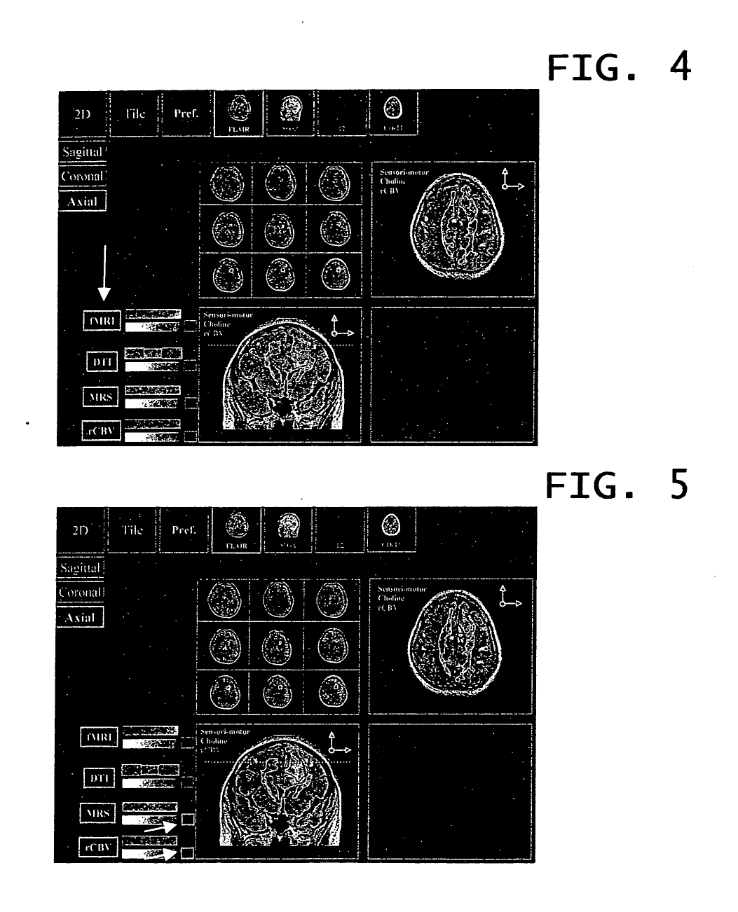

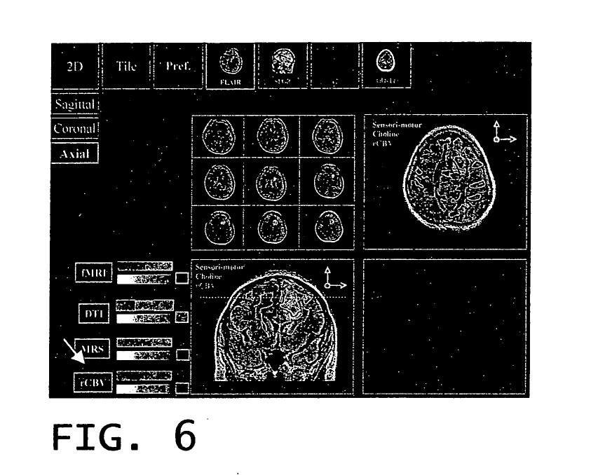

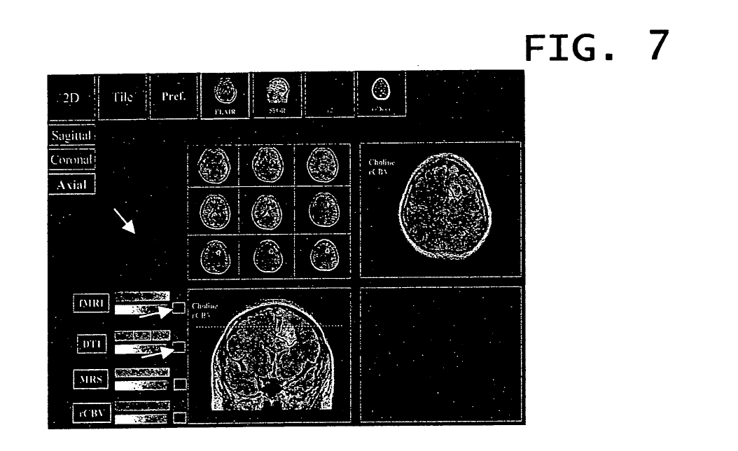

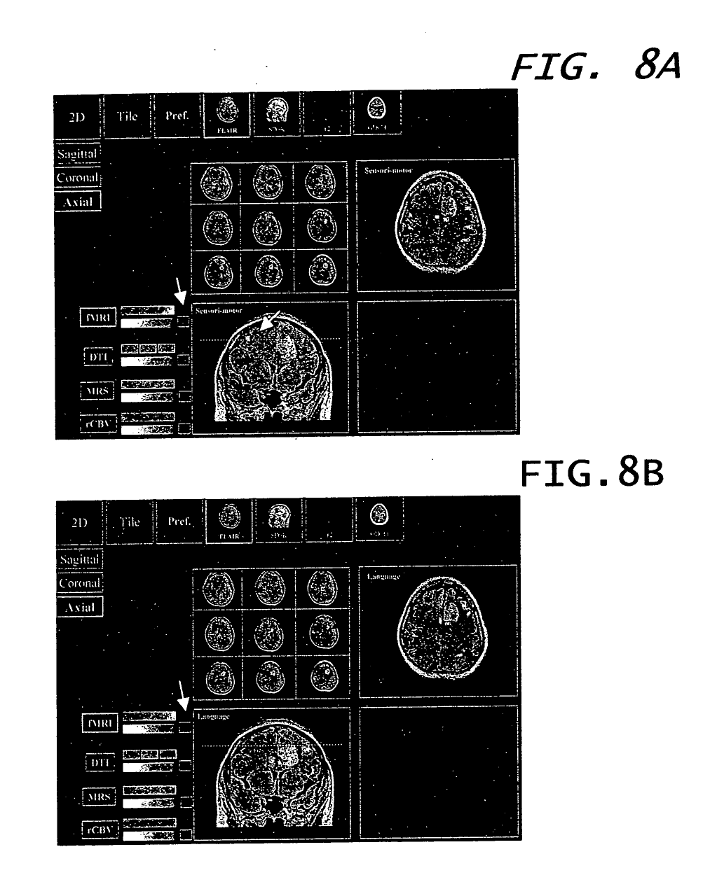

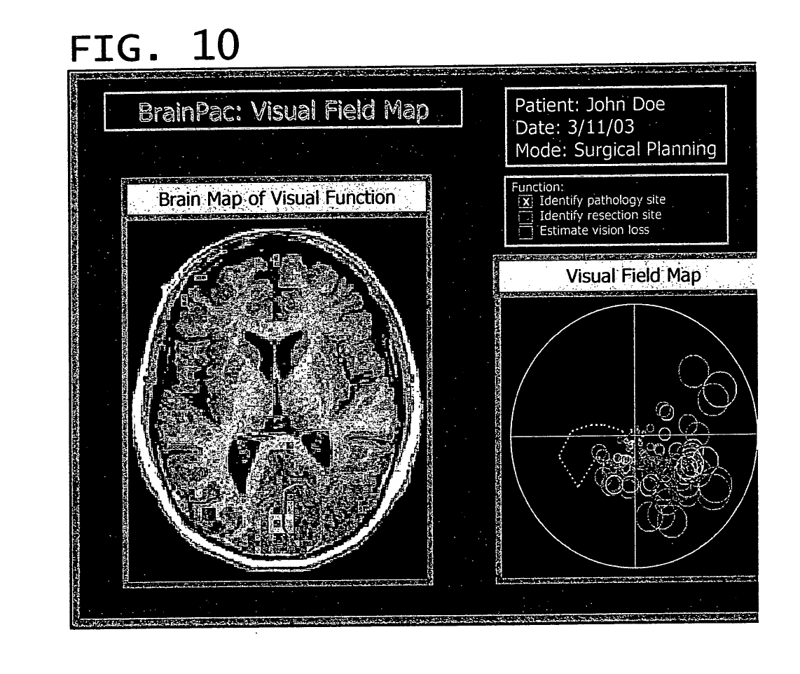

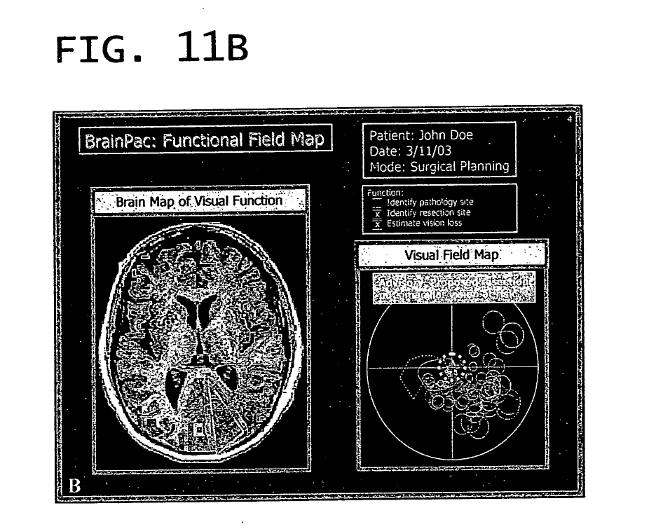

The invention is comprised of software which operates a video graphics display to integrate the spatial relationships of physiologic MRI or other imaging parameters, with anatomic and morphologic data present on standard images. FIGS. 1-11 show images produced on the display when the various tools and features of the present invention are used. All MRI or other image data are utilized in DICOM or other standard format. In the preferred embodiment images are divided into four types: functional magnetic resonance imaging (fMRI), Diffusion Tensor Imaging (DTI), Magnetic Resonance Spectroscopy (MRS), and perfusion imaging (which includes one or more of the following and its derivatives: cerebral blood volume, cerebral blood flow, vessel and tissue transit times and vascular morphology imaging). Other non-MRI generated physiologic data (including but not limited to PET-CT and CT-perfusion) can be integrated into the display as well. In general, any physiologically relevant data existing as images can be accommodated. By toggling each of these parameters on or off as shown in FIGS. 1-4, the relationships of the physiologic parameters in each of the image types to one another and to regional pathology can be determined and analyzed precisely for treatment planning. The ability to select or remove physiologic parameters that are not important or irrelevant allows clinicians to focus on key spatial relationships necessary for therapy.

Display of each of the four image types and their associated physiologic data can be controlled by one or more of the following options:

1. Threshold bars (FIG. 1) that allow one to select the sensitivity and specificity of a given physiologic data set.

2. Transparency bars (FIG. 1) that allow one to determine the relative transparency of a given data set in relation to other data sets in the brain, allowing one to visualize superimposed data sets without hindering the visualization of underlying normal brain (or other organ) tissue and pathology.

3. At the most transparent end of the transparency bar is a toggle box (FIG. 1, 5-7) that allows the outlining of a given physiologic parameter area in a color specific manner, such that outlines of each or all of the physiologic data sets can be seen with full visualization of the underlying normal tissue and pathology.

4. Boolean (logical) image mapping tools that allows display of areas of overlap or non-overlap of both anatomic and physiologic data.

5. A second toggle function (FIG. 1, 8, 9) for each physiologic parameter allows one to switch between various subcomponents of a given physiological data component. For example,
a. One can select among motor, language, and vision activation to determine the spatial relationships between eloquent cortex processing various functions to a given pathology.
b. Diffusion Imaging data can be selected such that a given white matter pathway can be selected and utilized in conjunction with fMRI or other physiologic data to map functional networks. DTI data subcomponents reflecting tumor extent and invasion can be displayed as well.
c. A toggle function can be selected to display various metabolite and metabolite ratio maps of interest. Tumor growth vectors, growth predictors or other reflections of tumor biology can be generated as other subcomponents of MRS or other physiologic data.
d. Likewise, a toggle function can be selected to display specific cerebral perfusion parameters including total blood volume, microvascular blood volume, vessel diameter, vascular and tissue transit times, along with the mean and distributions of each, as a reflection of tumor biology and characterization of neovascularity.

7. A drawing function is also provided that allows tracing of a lesion or other relevant feature in one orientation plane (i.e. multiple axial planes) to be viewed from planes in other orientations. This allows an analysis of the relationship of any given border of a lesion to any functional/physiologic parameter of interest in all planes in 3 dimensions.

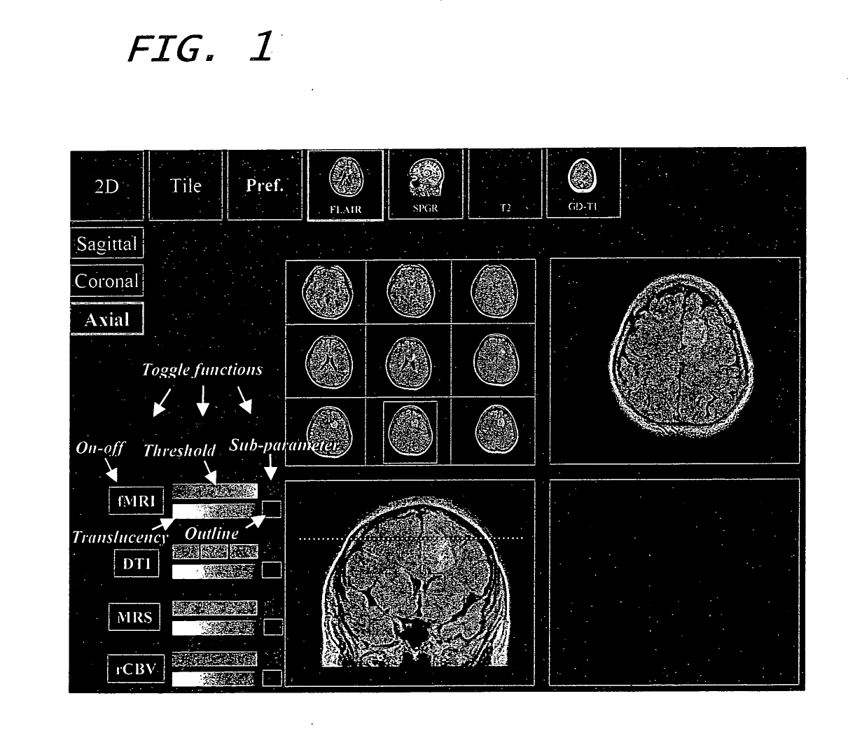

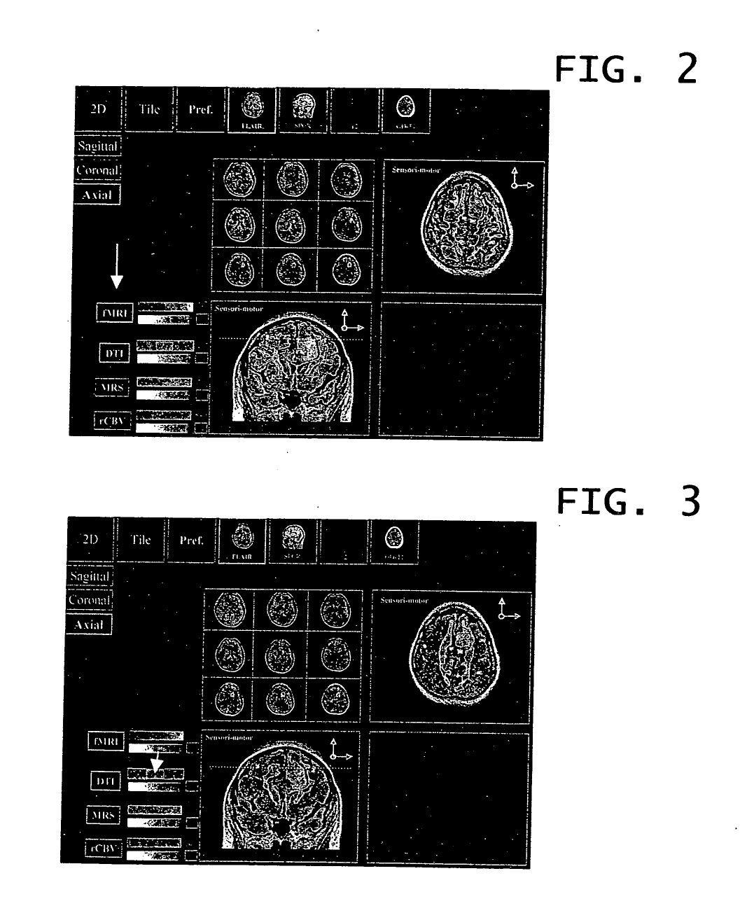

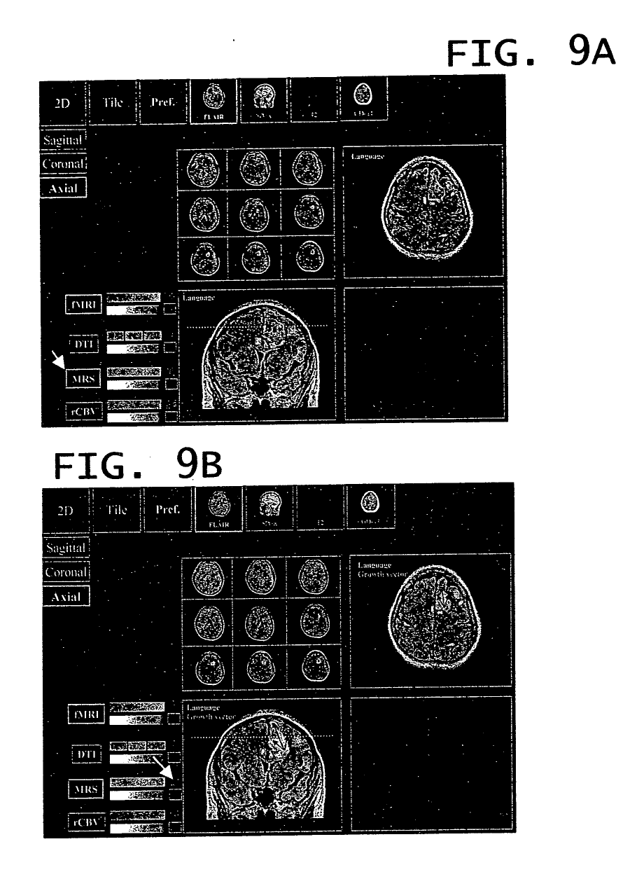

8. Each physiologic data set or subcomponent can be color-coded in the current field of view and overlaid on the anatomic and morphologic data (FIG. 2-9). For example, turning on the fMRI function can label in the upper left hand corner of the field of view the specific functional task that is being represented, with the text color matching that of the functional data set on the image. Alternately, turning on the diffusion tensor imaging parameter can be associated with a key that illustrates the color-coded anisotropy of white matter orientations (FIG. 3). Likewise, MR Spectroscopy and perfusion parameters can be associated with descriptions in the field of view, with the text matching in color to the respective physiologic data sets on the image. Physiologic parameter subcomponents can also be appropriately labeled within the field of view (FIG. 8), thereby facilitating the ease of integrating multiple data sets and multiple sub-components of the data for the purposes of diagnosis, treatment planning and patient management.

In addition to the two-dimensional display described above, other, more advanced features may also be employed. These include the display of Three-dimensional rotatable views of the brain or other organ, as well as multiplanar cutaway capabilities that can be used to image the relationship between deep tissues and pathology and physiologic maps.

When appropriate, four-dimensional data sets (3D+time) can also be displayed as movies of changing function, anatomy, or pathology evoked by different behavioral test conditions or from multiple imaging scan sessions, thereby allowing longitudinal assessment of disease progression/recovery/response to therapy. Within this mode, a menu provides other physiologic indices, such as magnetic resonance spectroscopy indices of tumor biology, cerebral perfusion indices that reflect tumor grade, grade conversion and tumor recurrence, and functional field maps of the visual system, sensori-motor system, or language system.

The system provides the ability to assess the quality of physiologic data, such as the impact of lesion-induced neurovascular uncoupling, and the quantification of hemispheric dominance when supplied with appropriate imaging and behavioral data sets.

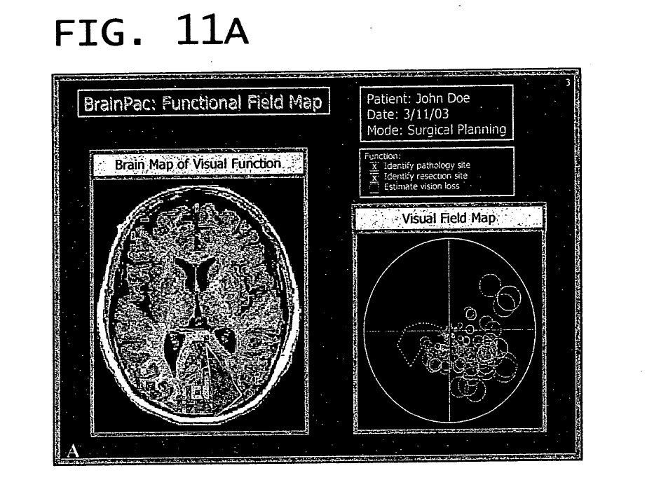

The system provides a virtual treatment mode in which the clinician can explore a variety of invasive therapeutic options (surgery, radiation, etc.) and estimate their potential impact on patient outcome prior to the actual treatment (FIG. 10, 11). In essence, this constitutes an MRI-based system for image-guided-treatment of tumors and other focal pathologies. A unique display technology allows the physician to simultaneously view brain function in an anatomical context and, for vision, in the context of a map of the patient's visual field (similar in format to a conventional Humphrey visual field exam). Standard imaging data as well as unique physiological imaging data (i.e MRS and RCBV) are used together to chart tumor extent and identify important differences in tumor biology throughout the pathology site. Using these interactive and interdependent displays, the physician identifies pathological tissue that will be surgically removed or irradiated. Once identified, the targeted tissue is “virtually” removed and the functional displays are updated in order to show how the proposed treatment may alter the patient's neurological function. In short, the system provides a suite of interactive tools to assist the physician in selecting the most appropriate treatment options to optimize therapeutic effect while maximally preserving brain function and quality of life.

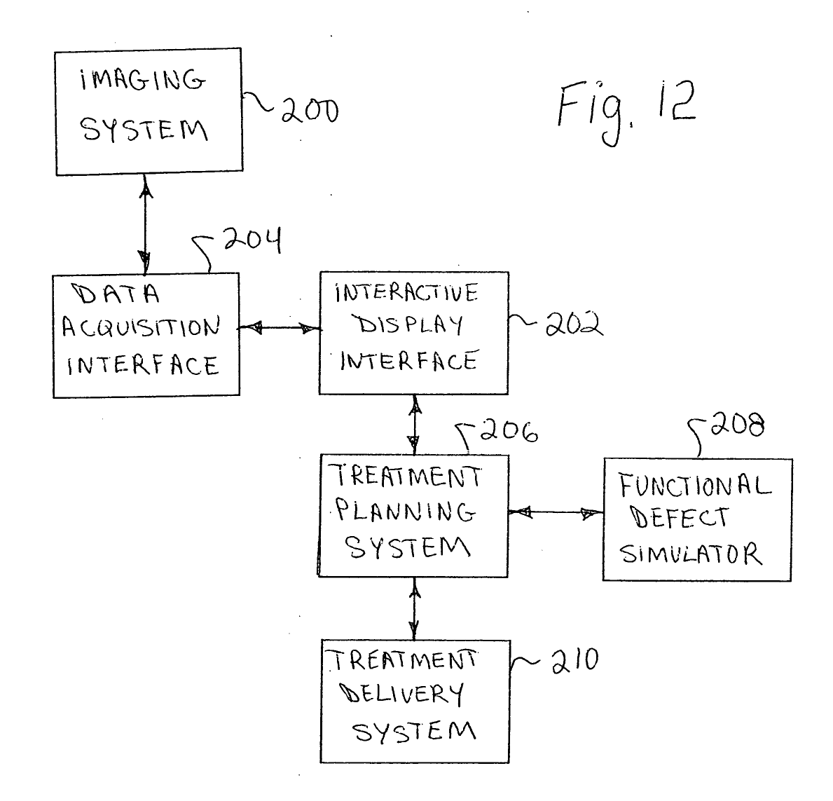

Referring particularly to FIG. 12, a system which employs the present invention includes a number of functional software modules that together provide the above-described functions. In the preferred embodiment an MRI imaging system 200 is employed as described above, but other imaging systems may also be used. The central functional module is an interactive display interface 202 which produces the above-described display screens and responds to the various toggles, buttons and sliders depicted on the display. The four types of images controlled by the toggle switches reside on four respective overlapping display layers plus a background layer which depicts an anatomical image of the brain.

Images are imported to each display layer of the interactive display interface 202 by making requests to the data acquisition interface 204. The data acquisition interface 204 may simply request stored DICOM images which were previously acquired, or it may enable the user to prescribe a new scan to acquire an image of any of the four types. In the case of a request for an fMRI acquisition the data acquisition interface 204 not only prescribes the scan, but also the stimulation sequences associated therewith. The delivery of contrast agent as part of a perfusion imaging procedure can also be prescribed through the data acquisition interface 204. The data acquisition interface also processes the acquired image data to produce the requested information using fMRI, DTI, perfusion imaging or MRS software in the known manner.

The interactive display interface 202 also includes registration and scaling software tools that insure images used in the displayed layers are anatomically aligned and displayed at the same scale. These tools may or may not be used on a particular image being imported to the interactive display interface 202 depending on its source. When images are imported from different imaging systems or modalities, it is likely that they will have to be registered with the images in the other layers so that they are anatomically aligned with each other regardless of the view angle (in the case of 3D images).

Using the interactive and interdependent displays, the physician identifies pathological tissue that will be surgically removed or irradiated. Once identified, the targeted tissue is “virtually” removed using a treatment planning system 206 and the displays are updated in order to show how the proposed treatment may alter the patient's neurological functions. Updated field maps that indicate the altered neurological functionality are then used to drive a Function Defect Simulator 208 whereby the patient can experience a simulation or be made aware of neurological dysfunction potentially caused by the proposed treatment.

Finally, information from the Treatment Planning subsystem 206 can be passed to automated treatment delivery subsystems 210 such as those for targeted radiation or localized surgical therapy.

Next to loss of life, losses of visual, motor and language functions due to brain pathology and invasive treatment constitute a major concern for physicians and patients. The present invention provides powerful new tools to address these concerns.

1. A method for planning treatment of a brain disease, the steps comprising:
a) acquiring a plurality of different images of the brain which depict a corresponding plurality of different physiological parameters; and
b) displaying a plurality of said different images in overlapping manner such that their physiological parameters are spatially registered with each other.

2. The method as recited in claim 1 in which said different images include two or more of the following types:
functional magnetic resonance imaging (fMRI);
diffusion tensor image (DTI);
perfusion image; and
magnetic resonance spectroscopy image.

3. The method as recited in claim 1 which includes:
c) simulating a treatment of the disease.

4. The method as recited in claim 1 which includes:
d) controlling a treatment delivery system.

5. An interactive display interface which comprises:
means for importing medical images of a plurality of different types;
means for displaying on a different layer an imported medical image of each of the plurality of different types;
means for registering the displayed medical images such that they are spatially aligned with each other; and
means for electively displaying or not displaying an image in each layer.

6. The interactive display interface as recited in claim 5 which includes means for controlling the transparency of images in each of said layers.

7. The interactive display interface as recited in claim 5 in which the different types of images include:
functional magnetic resonance image;
diffusion tensor image;
perfusion image; and
magnetic resonance spectroscopy image.
